(19) United States
(12) Patent Application Publication
(10) Pub. No.: US 2024/0277431 A1
(43) Pub. Date: Aug. 22, 2024

(54) METHOD FOR CALIBRATING A MICROSURGICAL INSTRUMENT OF A TELEOPERATED ROBOTIC SURGERY SYSTEM AND RELATED SYSTEM

(71) Applicant: MEDICAL MICROINSTRUMENTS, INC., Wilmington, DE (US)

(72) Inventors: Matteo TANZINI, Pisa (IT); Michael John PROCTOR, Pisa (IT); Giuseppe Maria PRISCO, Pisa (IT); Massimiliano SIMI, Pisa (IT)

(21) Appl. No.: 18/570,380

(22) PCT Filed: Jun. 16, 2022
(86) PCT No.: PCT/IB2022/055584

§ 371 Date: Dec. 14, 2023

(30) Foreign Application Priority Data
Jun. 17, 2021 (IT) 102021000015899

(51) Int. Cl. A61B 34/35; A61B 17/00; A61B 34/00; A61B 34/30

(52) U.S. Cl. CPC A61B 34/35; A61B 17/00; A61B 2017/00725; A61B 2034/306; A61B 2034/715

(57) ABSTRACT

A method calibrates a surgical instrument of a teleoperated robotic surgery system. The surgical instrument has transmission elements associated with respective tendons and connected to an articulated end-effector connectable, to determine correlate movements between the transmission elements and articulated end-effector. Motorized actuators operatively connect to respective transmission elements to impart movement. The articulated end-effector is arranged and locked in a predetermined reference position univocally associated with a respective resulting position of each transmission element. The actuators are then actuated so each actuator contacts a respective transmission element and the position of the actuators when each actuator contacts a respective transmission element is stored, and the stored actuator reference positions are univocally associated with the end-effector reference position. A kinematic zero condition associates the stored actuator reference position with a virtual zero point. The actuating applies force less than or equal to a threshold force on the respective transmission element.

### TECHNOLOGICAL BACKGROUND OF THE INVENTION

### Field of Application

The present invention relates to a method for calibrating a microsurgical instrument of a teleoperated robotic surgery system.

Therefore, the present description more generally relates to the technical field of operational control of robotic systems for teleoperated surgery.

### Description of the Prior Art

In a teleoperated robotic surgery system the actuation of one or more degrees of freedom of a slave surgical instrument is generally enslaved to one or more master control devices configured to receive a command imparted by the surgeon. Such a master-slave control architecture typically comprises a control unit which can be housed in the robotic surgery robot.

Known hinged surgical instruments for robotic surgery systems include actuation tendons or cables for transmitting motion from the actuators, operatively connected to a backend portion of the surgical instrument, distally to the tips of the surgical instrument intended to operate on a patient anatomy and/or to handle a surgical needle, as for example shown in documents WO-2017-064301 and WO-2018-189729 in the name of the same Applicant. Such documents disclose solutions in which a pair of antagonistic tendons is configured to actuate the same degree of freedom as the surgical instrument. For example, a rotational joint of the surgical instrument (degree of freedom of pitch and degree of freedom of yaw) is controlled by applying tensile force applied by the torque of the aforesaid antagonistic tendons.

Further known are surgical instruments in which the same pair of tendons is capable of simultaneously actuating more than one degree of freedom, such as shown in WO-2010-009221 in which only two pairs of tendons are configured to control three degrees of freedom of the surgical instrument.

For example, US-2020-0054403 shows an engagement procedure of a surgical instrument at an actuation interface of a robotic system, in which motorized rotary disks of the robotic system engage with corresponding rotary disks of the surgical instrument in turn connected to actuation cables of degrees of freedom of the end-effector of the surgical instrument. The engagement procedure described therein allows recognizing whether the surgical instrument is operatively engaged with the robotic system, evaluating the response perceived by the motorized rotary disks of the robotic system.

Typically, tendons for robotic surgery are made in the form of metal cords (or strands) and are wound around pulleys mounted along the surgical instrument. Each tendon can be mounted on the instrument and elastically preloaded, or pre-conditioned prior to assembly on the instrument, so that each tendon is always in a tensile state in order to provide a rapid actuation response of the degree of freedom of the surgical instrument when activated by the actuators and, consequently, to provide good control over the degree of freedom of the surgical instrument.

In general terms, all the cords are subject to elongation when subjected to loads. New cords of the intertwined type typically have a high elongation of plastic-elastic nature when under load due at least in part to the unraveling of the fibers forming the cord.

For this reason, before assembly on the surgical instrument, it is common practice to subject the new tendons to a high initial load in order to remove the residual plasticity of the drawing and intertwining process or of the material itself.

In general, the cords typically have three lengthening (elongation) elements:
(1) elastic elongation deformation, which is recovered when the tensile load stops;
(2) recoverable deformation, i.e., a relatively small deformation which is gradually recovered over a certain period of time and is often a function of the nature of the intertwinement, and can take a period of time between a few hours and a few days when not subjected to any load;
(3) non-recoverable permanent elongation deformation.

The permanent elongation deformation, as described above, can be achieved by a cord breaking-in procedure, performed prior to assembly on the instrument, which can comprise loading and unloading cycles and involve a plastic elongation deformation of the fibers themselves.

Viscous creep deformation under tensile load is a time-dependent effect which affects some types of intertwined cords when subject to fatigue and can be recoverable or non-recoverable typically depending on the intensity of the applied load.

Generally, the fatigue behavior of polymer fibers differs from the fatigue behavior of metal fibers in that the polymer fibers are not subject to crack propagation breakage, as instead are metal fibers, although cyclic stresses can lead to other forms of breakage.

WO-2017-064306, in the name of the same Applicant, shows a solution of an extremely miniaturized surgical instrument for robotic surgery, which uses tendons adapted to support high radii of curvature and at the same time adapted to slide on the surfaces of the rigid elements, commonly referred to as “links”, which form the hinged (i.e., articulated) tip of the surgical instrument. In order to allow for such a sliding of the tendons, the tendons-link sliding friction coefficient must be kept as low as possible, and the above-mentioned document teaches to use tendons formed by polymer fibers (rather than using steel tendons).

Although advantageous from many points of view, and indeed as a consequence of the fact that an extreme miniaturization of the surgical instrument is obtained by virtue of the use of the aforesaid tendons formed by polymeric fibers, in the context of this solution it becomes even more important to avoid the occurrence of an elongation or a shortening (contraction) of the tendons under operating conditions of the surgical instrument, because with the same variation in length, as the size decreases, the uncontrollability effects of the miniaturized surgical instrument would be accentuated.

Metal tendons have a modest recoverable elongation and the aforementioned preloading processes performed before assembly on the surgical instrument are typically sufficient to completely remove the residual plasticity, while the preload to which they are subject when assembled maintains an immediate reactivity in use.

Otherwise, the tendons made of polymer materials have high elongations due to the contributions described above; moreover, the preloading processes, if carried out before assembly, do not prevent the tendon from quickly recovering a large fraction of the recoverable elongation as soon as the tendons are subject to low tensile loads. If on the one hand the forecasting of any high assembly preloads prevents the recovery of the deformation, on the other hand it aggravates the creep process of the polymer tendon even when not in use, forcing the tendon to stretch almost indefinitely and weaken, and therefore is not a viable strategy.

For example, intertwined cords formed by high molecular weight polyethylene fibers (HMWPE, UHMWPE) are usually subject to non-recoverable deformation, while intertwined cords of aramid, polyesters, liquid crystal polymers (LCP), PBO (Zylon®), nylon are less affected by this feature.

In the case of surgical instruments, the variation in the length of the tendons attributable to the tendon elongation phenomenon described above, as well as the recovery of the elongation, is highly undesirable, in particular when under operating conditions, because it would necessarily impose objective complications in the control in order to maintain an adequate level of precision and accuracy of the surgical instrument itself.

In particular, for miniaturized instruments in which the accuracy of the robotic motion of the articulated end-effector is also a fundamental element in determining clinical performance, the actuation of tendons even of a few tens of microns (μm) can determine a rotation of some degrees of the articulated termination (e.g., hinged wrist, as shown for example in WO-2017-064301).

An example of a tendon actuation system comprising a robotic manipulator comprising a motorbox having motorized linear actuators and a surgical instrument having a proximal interface portion (or backend portion) comprising corresponding transmission pistons of the motion imparted by the motorized actuators to respective actuation tendons is shown for example in WO-2018-189729 in the name of the same Applicant.

However, the manufacturing methods but above all assembly methods of such miniaturized instruments make the repeatability of such an assembly extremely difficult, characterizing an intrinsic variability in the position of the motion transfer means, discs or pistons, with respect to the central kinematic zero position of the articulated end-effector.

For miniaturized instruments in which the position of the backend actuation means are not uniquely associated with a known position of the end-effector between one instrument and another, it is impossible to define the kinematic zero or reference or “kinematic zero point” position with the common engagement means.

In fact, given a kinematic zero position of the articulated end-effector, each instrument will have a different position of the backend actuation means such as discs or pistons and such a diversity is significant and not negligible. In such cases, it would therefore not be acceptable to advance the motors to a known engagement position as generally known in the art since the zero position is different between one instrument and another.

Furthermore, if the surgical instrument is provided with tendons designed to slide on surfaces of the end-effector with minimal friction as polymer fiber tendons, it would not even be acceptable to rely on the non-extensibility of the tendons, carrying them under load as would be the case with non-extensible steel tendons, since the tendons are polymeric and they would deform in a manner which is difficult to predict. In other words, it would necessarily be unpractical to preload such tendons until the expression and detection of a high resistant force (BEMF) as for example shown in US-2020-0054403 because in the event of polymeric tendons they could be subject to heavy plastic deformation.

US-2021-137618 of the same Applicant shows a solution of a robotic system for surgical teleoperation having a system for transmitting actuating forces to the surgical instrument comprising motorized pistons which linearly advance to come into contact with respective counter-pistons of the surgical instrument through a sterile barrier. The counter-pistons in turn stress the polymer actuation tendons of the degrees of freedom of the articulated tip of the surgical instrument. Polymeric actuation tendons are shown, for example, also in US-2020-008890.

For example, US-2020-054403 shows a calibration method which includes locking the tip of the surgical instrument.

For example, US-2021-0052340 shows a calibration process of the surgical instrument which incudes bringing a degree of freedom of the tip of the articulated surgical instrument to hit against the inner wall of a cannula fitted thereon in two opposite directions, so as to calculate the average position and store it as a reference position of that degree of freedom.

US-2018-214219 shows a surgical instrument provided with a toothed device for locking the degrees of freedom of the articulated tip of the instrument without touching it. Such a device can be inserted while the instrument is in use and is advanced along the insertion cannula of the instrument, if necessary, to reach the articulated end of the instrument in the operating field.

Therefore, in brief, the need is felt to precisely define the “kinematic zero point” in a precise and timely manner for each surgical instrument.

In particular, there are needs to precisely define the “kinematic zero point” in the case of surgical instruments having a miniaturized end-effector, and further in the case of surgical instruments actuated by antagonistic polymeric cables, and also in the case of surgical instruments made with wide production variability.

### SUMMARY OF THE INVENTION

It is the object of the present invention to provide a method of calibrating a surgical instrument of a robotic surgery system, which allows overcoming at least partially the drawbacks complained above with reference to the background art, and to respond to the aforementioned needs particularly felt in the technical field considered. Such an object is achieved by a method according to claim 1.

Further embodiments of such a method are defined by claims 2-26.

It is further the object of the present invention to provide a robotic surgery system capable of performing and/or adapted to be calibrated by the aforesaid method of calibrating a surgical instrument. Such an object is achieved by a system according to claim 27.

Further embodiments of such a system are defined by claim 28.

More in particular, it is an object of the present invention to provide a solution in line with the aforesaid technical requirements, with the features summarized below.

It is a further particular object of the present invention to provide a method which, before the teleoperation, is capable of matching a single configuration of the plurality of motors (e.g., six motors) belonging to the motor equipment (or “motorbox”) of the aforesaid robotic platform, to a single configuration of the surgical instrument consisting of at least two degrees of freedom belonging thereto (e.g., the degrees of freedom referred to as “pitch” and “yaw”).

The kinematic zero point is given by the coupling of the position of the motorized actuators (i.e., the motors belonging to the motorbox) of the robotic manipulator and the position of the transmission elements (e.g., the pistons) of the surgical instrument.

The starting position of the motors is unique for the machine, i.e., the robotic manipulator or the robotic arm containing the motorbox housing.

The initial position of the pistons, on the other hand, can be unique for each surgical instrument.

While the variability of the motors is much more limited as the robotic manipulator, i.e., the robotic arm, is not a disposable element, and is associated with the machine and the life cycle thereof, the variability of the surgical instrument is much higher as the instrument is a disposable element, and can be changed with great probability after each teleoperation session.

Both the motorbox and the instrument have a unique configuration, which, for the motorbox, can for example be due to mounting imperfections.

Due to the extreme miniaturization of the instrument and the geometries of the actuation system, any type of difference from a hypothetical unique configuration, albeit a few cents of a millimeter, can have a large impact on the kinematic congruence between master device and slave device which affects operation during teleoperation.

Due to these drawbacks, to which are added the elastic-plastic deformations, recoverable and not, of the tendons, a teleoperation can be severely compromised. In fact, the position of the transmission elements, and therefore of the motorized actuators operatively connected thereto, associated with a known configuration of the end-effector is not perfectly repeatable due to small imperfections, such as the recoverable or non-recoverable elastic-plastic deformability of the polymeric tendons.

By virtue of the suggested solutions, it is possible to engage the instrument and carry out a “homing” operation, i.e., with known position of the end-effector, it is possible to reset the position of the actuators which are arranged on the transmission elements (e.g., pistons) of the surgical instrument in an always different manner.

By virtue of the suggested solutions, it is possible to engage the instrument and carry out a “homing” of the surgical instrument even if the transmission chain is designed to maintain extremely low friction (for example using polymeric tendons) and is thus characterized by requiring a very low actuation force to actuate a motion of the articulated end-effector.

The calibration procedure or method according to the present invention is preferably performed before each teleoperating step.

The calibration procedure contributes to the preparation for the teleoperation and can be performed after it has been verified that the surgical instrument is correctly engaged in the respective pocket of the robotic manipulator.

The calibration procedure can be performed after an initialization step comprising an initial conditioning step, in which the surgical instrument is subject to a conditioning (“pre-stretch”) of the tendons thereof, and before a teleoperating step.

The calibration procedure can be performed after an initialization step comprising an initial conditioning step, in which the surgical instrument is subject to a conditioning (“pre-stretch”) of the tendons thereof, and a holding step (“hold homing”) and before a teleoperating step.

The calibration procedure can be performed between two adjacent teleoperating steps, i.e., between the end of one teleoperating step and the beginning of the next teleoperating step. This occurs, for example, when during a teleoperating step, at least some of the polymeric tendons have undergone elongation deformation, and then the calibration procedure is performed so as to store an updated kinematic zero position before starting a subsequent teleoperating step.

For example, between two adjacent teleoperating steps an intermediate step can be interposed in which the surgical instrument of the slave device is not enslaved to the master device (i.e., the slave is not following the master), such as a suspended teleoperating step and/or a limited teleoperating step and/or an accommodation step and/or a rest step. The number of successive and adjacent teleoperating steps which can be performed during a teleoperated robotic surgery operation can depend on various contingent and specific needs.

In fact, during a teleoperating step in which the surgical instrument is completely enslaved to the master device, it can occur that the performance of at least some tendons undergoes degradation due to intensive actuation of the degrees of freedom of the surgical instrument, an actuation which can require the tendons to describe high radii of curvature (for example, with reference to degrees of freedom of pitch/yaw).

By virtue of the suggested solutions, it is possible to obtain and update the precise matching between the position of the motorized actuators of the robotic manipulator and the configuration of the end-effector of the surgical instrument, even where the tendons are subject to recoverable or non-recoverable elastic-plastic deformation, as well as where there is an intrinsic variability between one surgical instrument and another as a result of the extreme miniaturization of the end-effector.

By virtue of the suggested solutions, it is possible to lock the articulated tip of the surgical instrument by using a plug or cap, and the contact between each motorized actuator and the respective transmission element of the surgical instrument is detected by means of force sensors (load cells) of the motorized actuators. Therefore, it is not necessary to read the motor currents of the motorized actuators nor to use the motors themselves to lock the degrees of freedom of the hinged tip.

The provision of a constraining element in the form of a plug or cap which is fitted on the articulated tip of the instrument, abutting against said articulated tip on at least two opposite sides, allows locking one or more degrees of freedom of the articulated tip of the surgical instrument, avoiding any range of movements of the tip itself. Thereby, it is possible to lock the articulated tip in a desired known position, for example aligned with the longitudinal axis of the surgical instrument with a single position of the plug or cap (constraining element), making the calibration procedure quick and precise.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 shows in axonometric view a robotic system for teleoperated surgery, according to an embodiment;

FIG. 2 shows in axonometric view a portion of the robotic system for teleoperated surgery shown in FIG. 1;

FIG. 3 shows in axonometric view a distal portion of a robotic manipulator, according to an embodiment;

FIG. 4 shows in axonometric view a surgical instrument, according to an embodiment, in which tendons are schematically diagrammatically shown in a dashed line;

FIG. 5 diagrammatically shows the actuation of a degree of freedom of an articulated end-effector of a surgical instrument, according to a possible operating mode;

FIG. 6 is a diagrammatic sectional view of a portion of a surgical instrument and a portion of a robotic manipulator showing the actuation of a degree of freedom of a surgical instrument, according to a possible operating mode;

FIGS. 7A-D diagrammatically show a sequence of a calibration method, according to a possible operating mode;

FIG. 8 is a partially sectioned axonometric view for clarity showing an articulated end-effector of a surgical instrument, according to an embodiment.

FIGS. 9A and 9B diagrammatically show in section an articulated end-effector constrained by a constraining element, in which the tendons are not shown for clarity;

FIGS. 10A-D diagrammatically show a sequence of a calibration method, according to a possible operating mode;

FIGS. 11A-11C show details related to a sequence of interactions between motorized actuators, transmission elements and surgical instrument, according to an embodiment of the calibration method;

FIG. 12 shows a flow diagram of an embodiment of the calibration method;

FIGS. 13 and 14A-C and 15A-B show details related to respective sequences of interactions between transmission elements and surgical instrument, according to respective embodiments of the calibration method.

### DETAILED DESCRIPTION

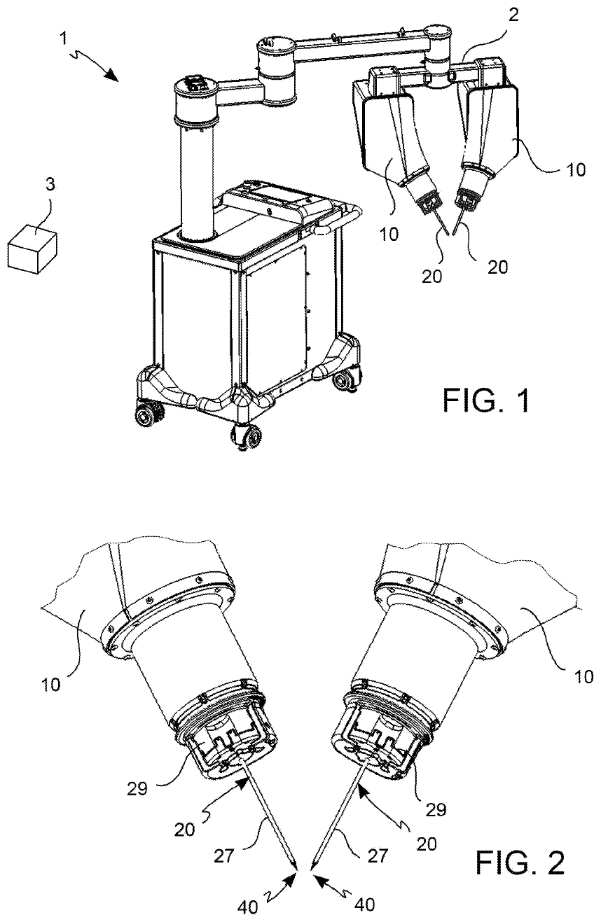

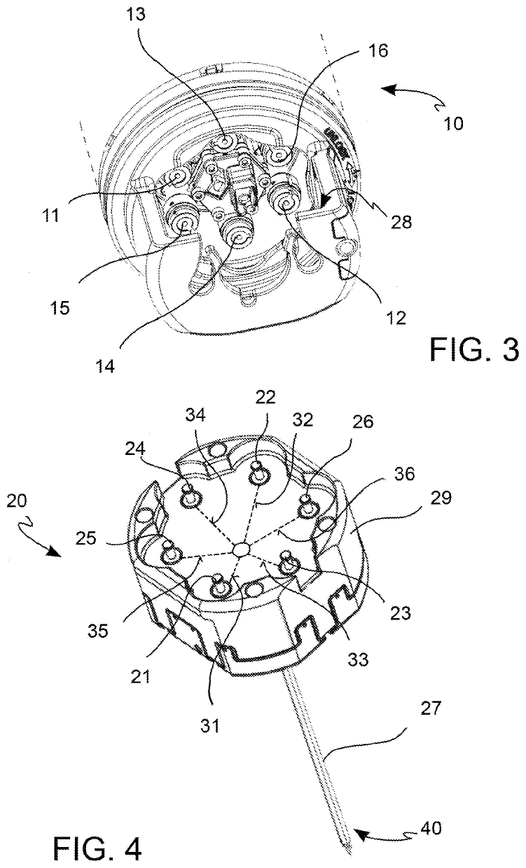

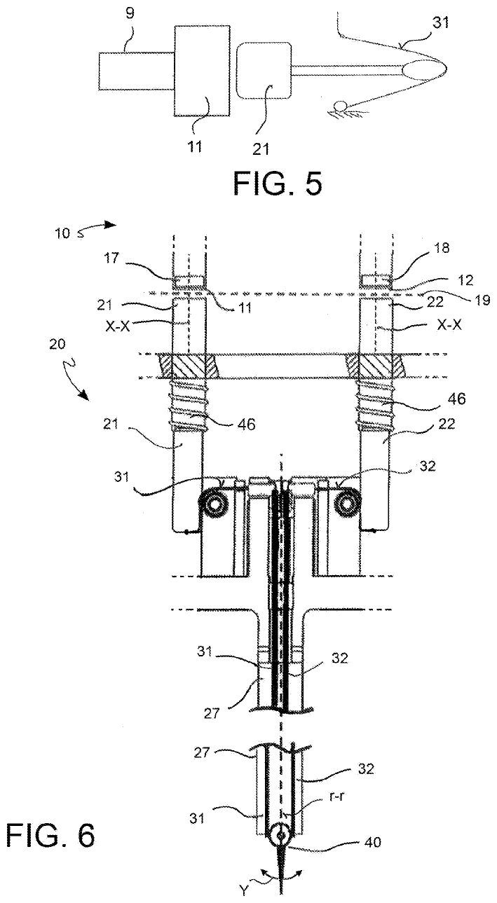

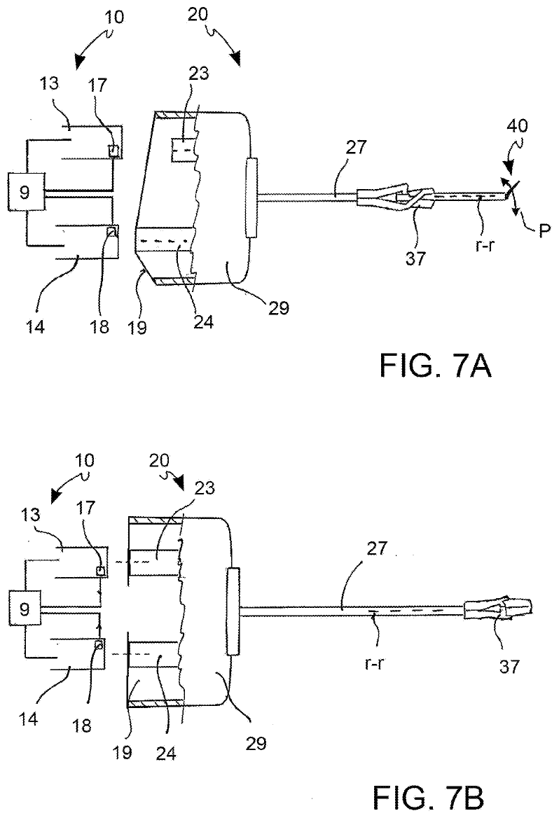

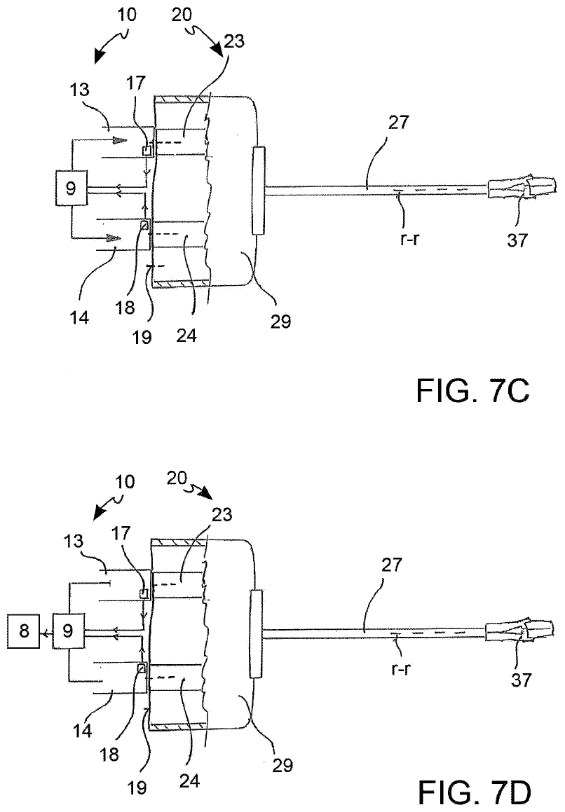

With reference to FIGS. 1-15, a method for calibrating a surgical instrument 20 of a teleoperated robotic surgery system 1 is described.

The surgical instrument 20 comprises a plurality of transmission elements 21, 22, 23, 24, 25, 26 associated with a respective plurality of tendons 31, 32, 33, 34, 35, 36, and an articulated end-effector device 40, which is mechanically connectable through respective tendons to the transmission elements, so as to determine a univocal correlation between a set of movements of the transmission elements and a respective movement or pose of the articulated end-effector device 40.

The teleoperated robotic surgery system 1 comprises, in addition to the aforesaid surgical instrument 20, a plurality of motorized actuators 11, 12, 13, 14, 15, 16 and control means 9. The motorized actuators 11, 12, 13, 14, 15, 16 are operatively connectable to respective transmission elements 21, 22, 23, 24, 25, 26 to impart movement to the transmission elements under control of the control means.

The method first comprises a step of arranging and locking the articulated end-effector device 40 in a predefined known position (which can in principle be any desired position as long as it is known and pre-designated for this purpose), considered as the reference position of the articulated end-effector device 40. Such a reference position of the articulated end-effector device 40 is univocally associated with a respective resulting position of each of the transmission elements 21, 22, 23, 24, 25, 26.

The method then provides the steps of actuating the motorized actuators 11, 12, 13, 14, 15, 16 so that each of the motorized actuators comes into contact with a respective transmission element 21, 22, 23, 24, 25, 26, and then storing the position of all the motorized actuators 11, 12, 13, 14, 15, 16 when each motorized actuator comes into contact with a respective transmission element, and considering the set of stored positions of the motorized actuators as a reference position of the motorized actuators univocally associated with the reference position of the end-effector device 40.

The method then comprises defining a kinematic zero condition, associating the aforesaid stored reference position of the motorized actuators a virtual zero point with respect to which the movements imparted by the control means 9 to the motorized actuators 11, 12, 13, 14, 15, 16 are (are to be) referred.

The aforesaid actuating step comprises controlling the motorized actuators 11, 12, 13, 14, 15, 16 so that they apply a force greater than zero and less than or equal to a threshold force on the respective transmission element of the surgical instrument.

With reference to the articulated end-effector device (which will also be defined hereinafter as a “hinged terminal” or “articulating tip” or “articulated end-effector”), it should be noted that it, in an implementation option, it is preferably a hinged wrist (i.e., cuff) having degrees of freedom of pitch, yaw and opening/closure (also referred to as “grip”), and preferably also a degree of freedom of rotation (also referred to as “roll”).

The method can be performed for example before using the surgical instrument.

According to an implementation option, the aforesaid step of actuating the motorized actuators 11, 12, 13, 14, 15, 16 comprises actuating the motorized actuators so that each of them comes into contact with a respective transmission element 21, 22, 23, 24, 25, 26), without moving it, or by slightly moving it to compensate for any deformation of the associated polymeric tendons.

According to an embodiment of the method, said threshold force is predetermined in a preliminary step of determining a threshold force, so as to impart a slight preload to the tendons operatively connected to both the transmission elements 21, 22, 23, 24, 25, 26 and to the articulated end-effector device 40, under conditions in which the end device 40 is held still and locked.

In such a case, the aforesaid actuating step comprises controlling the motorized actuators 11, 12, 13, 14, 15, 16 so that they apply a force equal to the aforesaid threshold force on the respective transmission element of the surgical instrument, within a tolerance E.

According to an embodiment, the method is applied to a teleoperated robotic surgery system comprising force sensors 17, 17′, 18, 18′, each of which is operatively connected to a respective transmission element 21, 22, 23, 24, 25, 26, and/or in which the motorized actuators 11, 12, 13, 14, 15, 16 are configured to apply force to respective transmission elements 21, 22, 23, 24, 25, 26 and detect the force actually applied on each transmission element.

In such a case, the aforesaid step of applying a force greater than zero and less than a threshold force on each transmission element 21, 22, 23, 24, 25, 26 comprises applying a force to the transmission element 21, 22, 23, 24, 25, 26 by means of a feedback control loop, in which the feedback signal is representative of the force applied to the transmission element as actually detected by the respective force sensor 17, 17′, 18, 18′ operatively connected to the transmission element or to the respective motorized actuator 11, 12, 13, 14, 15, 16.

According to a particular implementation option, in which the system comprises a sterile, slightly elastic drape 19 arranged between the actuators and the transmission elements, the force is applied by the motorized actuator on the respective transmission element (e.g., 21) through the sterile drape 19. In such a case, the force sensors 17, 17′, 18, 18′ mounted on the actuator (e.g., 11) detect the actuator-drape-transmission element contact force, and thus the contact between the actuator and the transmission element is in this case indirect. The sterile drape or cloth 19 is preferably elastically preloaded in a flat configuration thereof which results in a preload in a proximal direction on the bottom of the motorized actuators when the actuators advance. The force sensors 17, 17′, 18, 18′ are preferably on the bottom of the motorized actuators of the robotic manipulator 10, i.e., on the non-sterile side of the sterile drape 19.

In accordance with an implementation option, the articulated end-effector device 40 comprises joints, and the aforesaid predetermined known position of the articulated end-effector device 40 is a position corresponding to the condition in which each joint of the articulated end-effector device 40 is in a centered position of the joint workspace thereof.

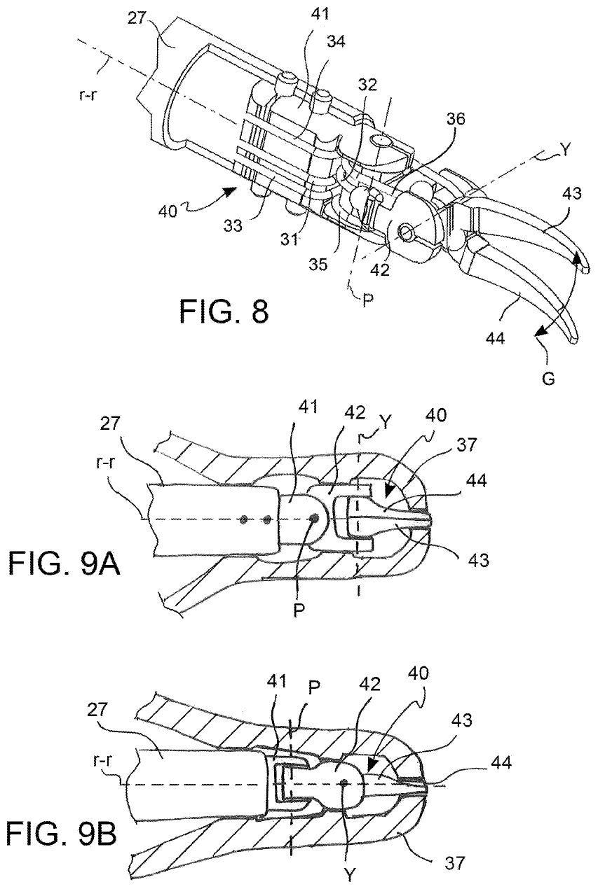

For example, in the implementation option shown in FIG. 8, rotational joints are used which define a degree of freedom of pitch P, a degree of freedom of yaw Y, and a degree of freedom of grip G, and the aforesaid centered position is a centered angular position.

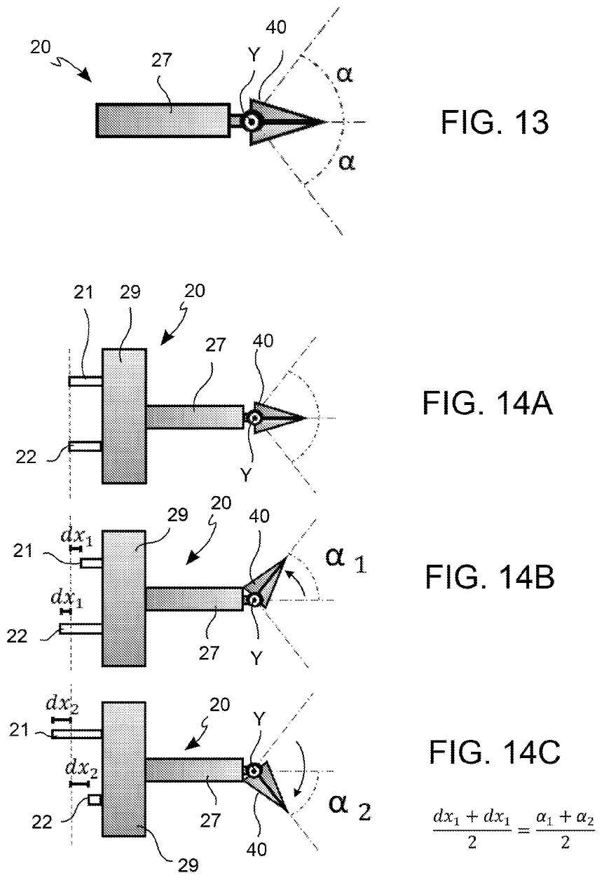

As shown for example in FIG. 13, the centered angular position can define for the rotational joint defining the degree of freedom of yaw Y of the end-effector 40 two angles α equal to each other between said centered angular position and the respective stroke ends.

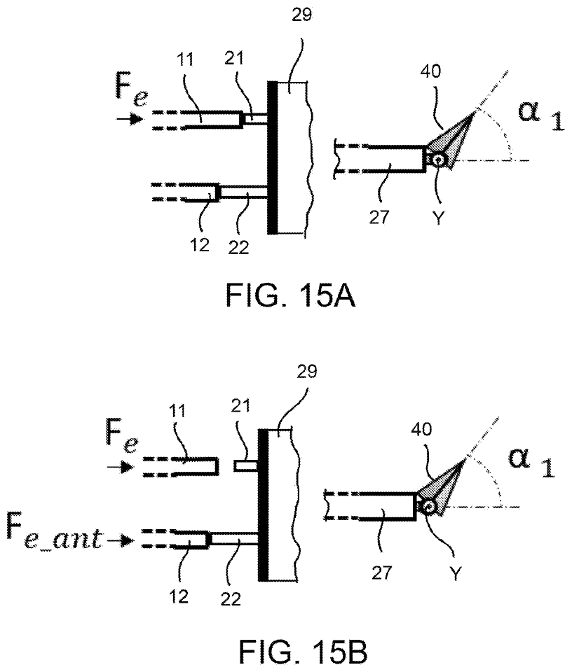

As shown for example in FIG. 14A-C, the degree of freedom of yaw Y is brought to the stroke end, acting on the antagonistic transmission elements 21, 22 describing first an angular distance α1 and then a second angular distance α2 (in the example shown here greater than the first angular distance), in which according to an implementation the zero point is calculated as the midpoint of the stroke carried out by the antagonistic transmission elements 21, 22 to describe said angular distances α1 and α2, according to the relationship:

$$dx 1 + dx 2 / 2 = α 1 + α 2 / 2$$

According to another implementation option, in which the articulated end-effector device 40 comprises joints, the aforesaid predetermined known position of the articulated end-effector device 40 is a position corresponding to the condition in which the articulated end-effector device 40 is aligned with the axis of a shaft 27 or rod 27 of the surgical instrument 20.

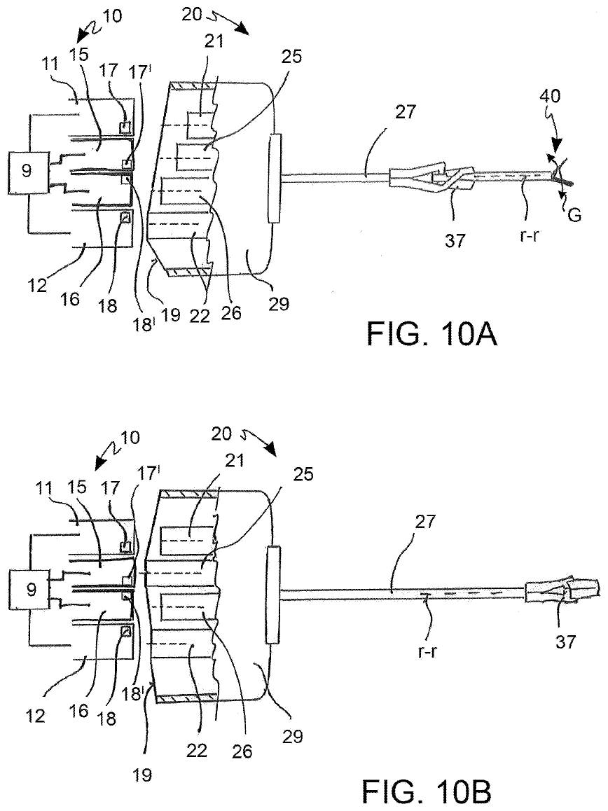

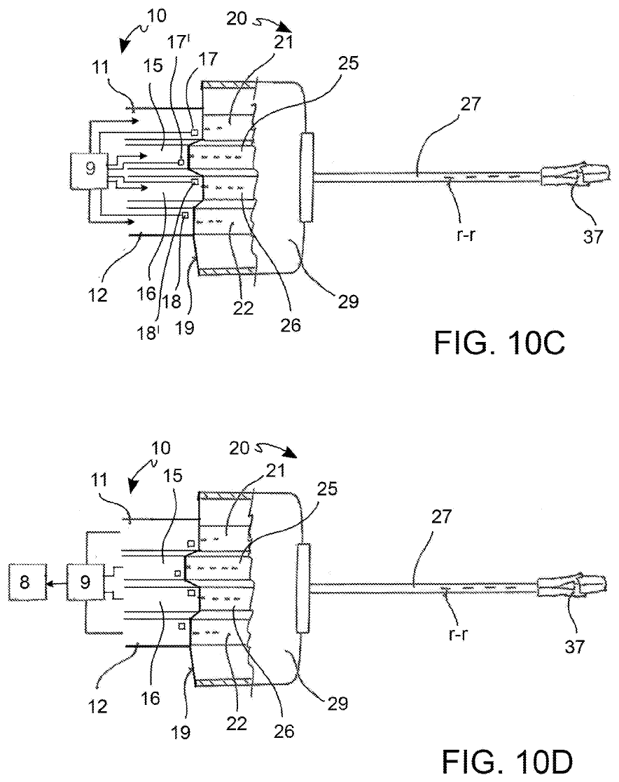

Preferably, the shaft is a rigid shaft extending along a longitudinal extension direction r-r (as shown in FIG. 10) so that the articulated end-effector device 40 is aligned with the longitudinal extension direction r-r of the shaft 27 and, preferably, the centered angular position of each rotational joint is aligned with said longitudinal direction r-r; thereby, the longitudinally squat or elongated body of the links (i.e., junction elements, i.e., connection elements) is longitudinally aligned with the shaft 27.

According to an embodiment of the method, the reference position of the articulated end-effector device 40 is held constrained by a tip cap 37. The tip cap 37 can be adapted to lock the degrees of freedom of pitch, yaw and grip, and can be adapted to also lock the degree of freedom of roll i.e., rotation around the longitudinal axis r-r.

According to an implementation option of the method, the aforesaid threshold force, at which the motors of the motorized actuators 11, 12, 13, 14, 15, 16 stop in contact with the respective transmission elements 21, 22, 23, 24, 25, 26 is in a range of 0.01 N to 5.0 N, preferably between 0.05 N and 2.0 N.

In accordance with an implementation option of the method, a control of the offset between the reference position of the motorized actuators 11, 12, 13, 14, 15, 16, and preferably of each of the motorized actuators, for example independently of the others, and a predetermined nominal zero position is carried out, and if such an offset is greater than a maximum allowable absolute offset dx_{MAX}, the calibration procedure is considered invalid.

According to an implementation option, it is sufficient that only one of the actuators has an offset greater than the aforesaid maximum absolute offset dx_{MAX}, to consider the calibration procedure invalid.

In accordance with an embodiment of the method, a control of the relative offset between the positions reached by each motorized actuator 11, 12, 13, 14, 15, 16 when in contact with the corresponding transmission element 21, 22, 23, 24, 25, 26 is carried out, and if such a relative offset is greater than a maximum allowable relative offset dx, the calibration procedure is considered invalid.

According to an implementation option, the relative offset between motorized actuators associated with the transmission elements of a pair of antagonistic transmission elements is controlled.

According to possible implementation options, the maximum allowable relative offset dx is in the range of 0 to 20.0 mm, and preferably between 5 and 15 mm.

In accordance with one embodiment of the method, one or more pairs of antagonistic transmission elements (21, 22), (23, 24), (25, 26) operatively connectable to respective one or more pairs of antagonistic tendons (31, 32), (33, 34), (35, 36) are provided. Each pair of antagonistic tendons is adapted to move a link (i.e., connecting element in a single piece) 42, 43, 44 of the articulated end-effector device 40 in opposite movement directions, e.g., in opposite angular directions, or, in other words, each pair of antagonistic tendons is adapted to move a respective degree of freedom (pitch P or yaw Y or grip G) in opposite directions.

According to an implementation option, elastic elements 46 are provided, which act on respective transmission elements 21, 22, 23, 24, 25, 26 to keep a constant minimum preload level adapted to space apart the transmission elements 21, 22, 23, 24, 25, 26 from the respective motorized actuators 11, 12, 13, 14, 15, 16.

In accordance with an embodiment, the aforesaid actuating step comprises controlling the motorized actuators 11, 12, 13, 14, 15, 16 so that, in a first contact step between motorized actuators and respective transmission elements, a first speed v1 is imparted to the motorized actuators and a first force F1 is applied on the respective transmission elements.

According to an implementation option, the actuating step comprises controlling the motorized actuators 11, 12, 13, 14, 15, 16 so that said first speed v1 is in a range of 0.1 to 30 mm/s, and preferably between 1 and 10 mm/s.

According to an implementation option, the actuating step comprises controlling the motorized actuators 11, 12, 13, 14, 15, 16 so as to stop the movement of said motorized actuators 11, 12, 13, 14, 15, 16 when the aforesaid first force F1 is detected to be in a range of 0.01 to 2 N, and preferably 0.05 N to 0.5 N.

In accordance with an implementation option, the actuating step comprises, in addition to the aforesaid first contact step, a retracting step, in which the motorized actuators 11, 12, 13, 14, 15, 16 retract by an offset dx1 (and a retracting speed v4), and a second advancement and second contact step, in which the motorized actuators 11, 12, 13, 14, 15, 16 advance with a second speed v2 and stop when a contact force equal to a second force F2 is detected.

According to an implementation example, the second force F2 is equal to the aforesaid threshold force.

According to an implementation option, said second speed v2 is lower than said first speed v1, and preferably in a range of 0.1 to 5 mm/s and preferably between 0.5 and 3 mm/s.

According to an implementation option, the aforesaid second force F2 is greater than said first force F1, and preferably in a range of 0.1 to 5N, and more preferably between 0.5 and 2 N.

In accordance with an implementation option, during the aforesaid retraction step, the movement of the motorized actuators is controlled so that the force applied by the latter reaches a third force value Fm.

According to an implementation example, the third force value Fm is preferably in a range of 0.1 to 5 N.

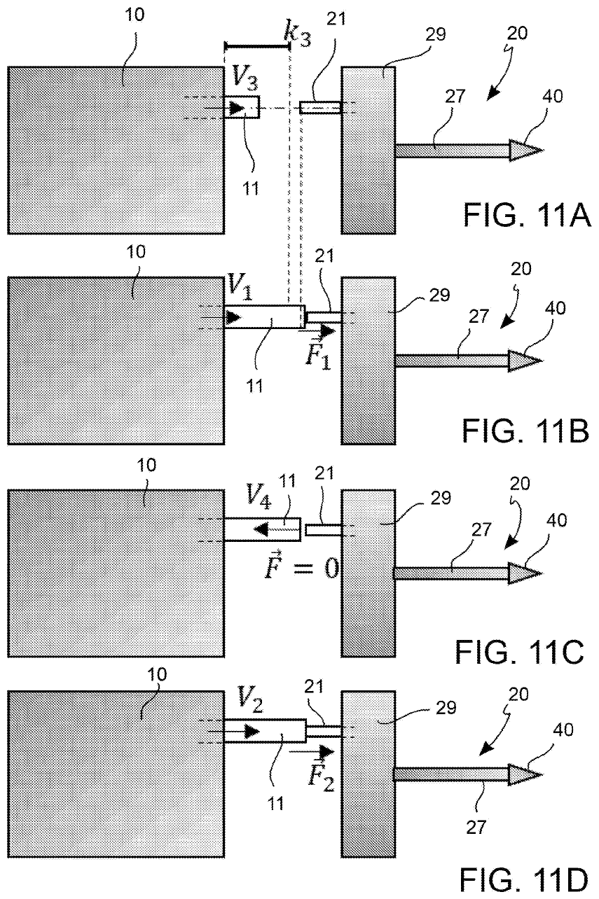

In accordance with an implementation option, the aforesaid actuating step comprises controlling the motorized actuators so that they advance with a speed equal to a third speed v3, greater than the aforesaid first speed v1 and second speed v2, when the position of the motorized actuators is in a predefined range (indicated as k₃ in FIG. 11A) in which the control means know that a free stroke regime is occurring, prior to the first contact with the transmission elements, along a space corresponding to a stroke dX3.

The aforesaid first speed v1, second speed v2 and third speed v3, and the retracting speed v4 are indicated in the implementation example shown in FIGS. 11A-D.

In accordance with an embodiment (already mentioned above) a flexible and elastic sterile drape 19 is interposed between the motorized actuators and the surgical instrument. In such a case, the force generated by the resistance of such a sterile drape is a known off-set or bias force Foff, and the control means 9 are configured to take into account, or to remove or not consider, such a known off-set or bias force Foff from the force checks carried out, and/or from the comparison with the threshold force.

According to an implementation option, the sterile drape 19 is elastic and is elastically deformed when in operating conditions. The elasticity of the drape 19 is aimed at bringing the cloth back into non-deformed flat configuration. Therefore, when the actuators advance to push, there is a minimum preload exerted by the drape 19 on the bottom of the actuators, while when an actuator retracts because it is pushed by the respective transmission element thereof, for example if the antagonist thereof is being pushed to the stroke end, the preload exerted by the drape is exerted on the transmission element and is directed distally.

In accordance with an embodiment of the method, the control means 9 move the articulated end-effector device 40, when it is in the condition to move without being locked by external constraints, by applying a maximum operating force (Fa), which is less than or equal to the aforesaid threshold force.

Such a maximum operating force is, in an implementation option, less than or equal to 5N.

According to an implementation option, the motorized actuators 11, 12, 13, 14, 15, 16 comprise pistons 11, 12, 13, 14, 15, 16.

In such a case, according to an implementation example, the tendons can be fixed, for example glued, to the respective piston (as shown in FIG. 6), which, therefore advancing along a straight path, defined by the piston, drags the glued end of the respective tendon. Returns (e.g., return pulleys) are provided in the backend 29 downstream of the piston (and upstream of the end-effector 40 and also of the shaft 27) which ensure that, when the piston advances, then the path of the tendon extends in the section upstream of the return, and therefore “pull” the respective degree of freedom to move it, carrying behind the other antagonistic tendon and therefore the other antagonistic piston.

In other words, when a piston is “pressed”, the degree of freedom is actuated in an angular direction, and the other antagonistic piston is “raised”.

According to an alternative implementation, the tendons are not glued to the piston but are glued to an inner wall of the instrument, and the advancing piston deflects the path of the tendon (like a guitar string), stretching it, itself acting as a return element.

According to another implementation option, the motorized actuators 11, 12, 13, 14, 15, 16 comprise rotary discs 11, 12, 13, 14, 15, 16.

Such rotary discs wind/unwind a proximal section of the tendon, moving by a certain angular displacement.

In such a case, the actuators are also preferably rotary discs which engage with the rotary disks of the transmission elements. Even the sterile drape, in such a case, can comprise rigid interfaces, for example inserts or hard plastic plates adapted to transfer a rotating actuating motion of the rotary discs.

The aforesaid rotary discs are, for example, capstans.

Two embodiments of the method are described below, both being applicable to when the antagonistic tendons are operatively connected (preferably directly fixed) to both respective transmission elements and to respective links of the articulated end-effector device 40 to actuate with opposite movements at least one degree of freedom (between the aforesaid at least one degree of freedom of the articulated end-effector device).

In a first of such two embodiments, the method provides that, after the contacting or engaging step between motorized actuators and transmission elements, the defining step is performed simultaneously on the antagonistic tendons of a pair of agonistic-antagonistic tendons for each degree of freedom of the end-effector device 40; furthermore, preferably, the aforesaid defining step is applied in succession to the various pairs of antagonistic tendons, i.e., it is performed for one pair at a time. In such a case, to lock a degree of freedom, both tendons of an antagonistic pair are appropriately stressed.

In a second of such two embodiments, the method provides that, after the contacting or engaging step between motorized actuators and transmission elements, the step of defining comprises, for each of the controlled degrees of freedom of the end-effector device 40:
bringing each of the degrees of freedom of the end-effector device 40 to a stroke end abutment;
applying a high force Fe to the respective transmission element, thus stressing the respective tendon;
storing, for each of the degrees of freedom, the corresponding position Xe of the transmission element which is thus obtained;
defining and/or recalculating the kinematic zero position based on the stored positions Xe of the transmission element for each of the degrees of freedom.

In such a case, preferably, the aforesaid bringing, applying, storing, and defining and/or recalculating steps are carried out for all the transmission elements, in particular for the transmission elements and the mutually antagonistic tendons, so that for each degree of freedom, the two positions (Xe, Xe_ant) of the two transmission elements associated with the antagonistic tendons of said degree of freedom are stored.

It should be noted that, in possible implementation options, the zero position is not necessarily halfway between the antagonistic abutments but depends on the shape and structure of the end-effector.

In accordance with an embodiment of the method, in which the angular distance between the kinematic zero position of a degree of freedom and the stroke end thereof is known, the defining step comprises:
bringing one degree of freedom of the end-effector device 40 to the end-of-stroke abutment,
bringing the force acting on a tendon of a pair of antagonistic tendons to a high force value Fe;
storing the position Xe of the transmission element corresponding to said tendon;
keeping the high force Fe applied to such a tendon, while the step of applying an antagonistic force Fe_ant on the other tendon of the pair of antagonistic tendons is carried out, in which such a high force Fe is greater than the aforesaid antagonistic force Fe_ant;
storing the position Xe_ant of the transmission element corresponding to the aforesaid antagonistic tendon;
calculating the kinematic zero position of the antagonistic transmission elements of such a pair of antagonistic transmission elements based on the stored values of the respective positions Xe, Xe_ant;
moving the aforesaid transmission elements to the calculated kinematic zero position.

According to possible implementation options, the method preferably includes repeating the steps described above for each degree of freedom, i.e., for each pair of antagonistic tendons, simultaneously or in succession.

According to a particular implementation option, shown in FIGS. 15A and 15B, the method provides engaging and preparing and adjusting the antagonistic tendons which act on the degrees of freedom of pitch and yaw, bringing them to a threshold force value lower than the high threshold value Fe, at which the end-effector device 40 of the surgical instrument does not move.

More specifically, being known the distance between an abutment position of an end-effector joint and the kinematic zero of the articulated wrist, a cable (or tendon) is moved bringing the joint in abutment, then a force is applied until reaching the high force value Fe and the corresponding position Xe of the piston is stored. Then the antagonistic cable (or tendon) is moved, by applying a force which reaches a value F_ant less than the high force value Fe, so that the degree of freedom of the end-effector does not move, and the corresponding position X_ant of the antagonistic piston is stored. Since the distance is known, the stored positions Xe and X_ant are used to calculate the kinematic zero position, and the pistons are finally arranged in such a kinematic zero position.

According to an implementation option, the method applies to when the aforesaid tendons are polymeric tendons, for example formed from intertwined or braided polymer fibers.

Such tendons change the lengthening thereof based on external parameters which cannot be controlled such as aging, temperature, preload, thus it is uncertain how elongated the cable is; precisely for this reason, it is particularly advantageous to perform the method described above.

According to an embodiment, the method applies to a robotic system consisting of a robotic system for micro-surgical teleoperation, in which the surgical instrument is a micro-surgical instrument.

Referring again to FIGS. 1-15, further illustrations of the surgical instrument to which the method of the present invention is applied will be provided below, useful for an even better understanding of the method itself, as well as further details, by way of non-limiting example, on some embodiments of the method.

According to an embodiment, the method comprises the following steps.
positioning the instrument in the special housing so that the coupling of the motorized actuators (or motors of the motorbox of the manipulator 10) is arranged with the transmission elements (pistons) of the surgical instrument. The motors of the motorbox must have previously been positioned in the zero position of the motorbox or in a configuration in which the motors have the motor shaft retracted;
moving the motors of the motorbox (even independently) in order to reach the pistons with a contact force F_{light}. Such a contact force F_{light} is the minimum force measurable by the force sensors positioned at the tip of the motor (for example, such a force corresponds to the aforesaid force greater than zero and less than or equal to a threshold force, applied to the transmission elements). The minimum applicable force must allow touching the piston without causing it to move. This is possible by virtue of the intrinsic friction of the piston coupling with the internal actuators of the instrument. Nevertheless, the degrees of freedom of the instrument are locked by a special cap which constrains the movement thereof to the initial position;
   activating the force control so as to hold the force on each piston and a minimum stress on the tendons;
   the coupling obtained is stored immediately before the first entry into teleoperation as kinematic zero and consists of the current position of the motors.

Preferably, the aforesaid positioning and moving steps can comprise the following steps.
(1) Command to set the kinematic zero position of the surgical instrument.

The command can be launched from one of two sources: an input from the user interface or an automatic input determined from the detection of the insertion of the surgical instrument.
(2) Procedure for setting the kinematic zero position of the surgical instrument.

The procedure for setting the kinematic zero position, also referred to as “Instrument Engagement”, is a sequence of software commands which move the motors of the motorbox to make the load cells engage with the pistons of the instrument. The zero position (i.e., kinematic zero) is set to be the position where all the pistons of the instrument are engaged with equal force, as measured by the load cells of the motorbox. To ensure the accuracy of the engagement and to obtain the completion of the engagement procedure in a short period of time, the engagement can occur through the repetition of a set of cycles, in which each cycle is a compromise between motor speed and distance and force until a sufficiently slow speed value and a final engagement force value are used for an accurate engagement which does not determine any movement of the tip of the end-effector.

The engagement routine receives a command to start the procedure for setting the zero position of the instrument. The routine verifies that the system state is ready and that the necessary sub-system initialization has been performed.

To reduce time, the routine commands a fast trajectory of the six axes of the motorbox to drive the pistons of the motorbox to a position close to the pistons of the instrument. Then, a speed value V_{MS} lower than the speed value of the aforesaid fast trajectory is imposed to obtain a first contact force F_{light} with the pistons of the instrument. Each axis stops independently when the respective load cell detects the contact force value F_{light}.

The axes are then controlled so as to touch the pistons of the instrument, thus determining a zero force. The contact force is then increased, in a programmed manner, up to the value which the load cells must have to be in the zero position.

For precise contact, the axes are controlled with a slow speed trajectory to contact the pistons and continue to move until a predefined specific force is obtained, and each axis stops independently when such a predefined force value Fₕₒₘₑ is reached on the respective load cell. When all the load cells detect the required forces and the movement of all the axes is stopped, the engagement procedure is completed.

If any of the axes does not detect the expected force value, in the respective load cell, at the distance allocated for the trajectory, the routine emits an error indication and forces the instrument to disengage.

In summary, therefore, the aforesaid procedure comprises:
a) checking the presence of the instrument;
b) checking that the motorbox axes have obtained the zero position thereof;
c) checking that the motorbox axes are in the zero backstop position thereof;
d) checking that the load cells have the respective calibration and offset values applied;
e) checking that the load cells have zero values within the limits to take noise into account;
f) loading the configuration parameters of the procedure:
   (i)) loading engagement force values:
      first contact force value
      engagement contact force value
   (ii) loading the engagement distances of the axes
      fast approach distance of the axes
      slow approach distance of the axes
      maximum allowable touch distance of the axes
   (iii) loading the engagement speed of the axes
      fast approach speed of the axes
      slow approach speed of the axes
      first engagement speed of the axes
      slow engagement speed of the axes
g) performing a quick trajectory to cover the interval between the rear position of the motorbox pistons and the instrument pistons:
   setting the contact force value;
   setting the speed value to the fast approach value;
   setting the distance value to the fast approach value;
   generating the fast approach trajectory with a trapezoidal speed profile using the set speed and distance;
   controlling the motorbox axes to move using the generated trajectory, in which the movement procedure comprises controls such that if a load cell reads a force greater than or equal to the contact force, the movement of that axis is stopped; the routine waits for all the axes to have completed the movement thereof; the distances not travelled of the excess trajectories are discarded;
   controlling the motorbox to move each axis which is in contact backwards to have a zero value from the load cell indicating no contact; this is done to ensure balanced contact in the next step;
h) performing the first contact trajectory with intermediate speed and touch:
   setting the contact force value;
   setting the speed value to the intermediate approach value;
   setting the distance value to the maximum allowed approach value;
   generating the fast approach trajectory with a trapezoidal speed profile using the set speed and distance;
   controlling the motorbox axes to move using the generated trajectory, in which each axis stops when the respective load cell force thereof is obtained; the routine waits for all the axes to have completed the movement thereof; the distances not travelled of the excess trajectories are discarded;
   controlling the motorbox to move each axis which is in contact backwards to have a zero value from the load cell indicating no contact: this is done to ensure balanced contact in the next step;
i) performing final contact, to have precise contact using slow speed and the required contact force:
   setting the contact force value to the value required for the zero position;
   setting the speed value to the slow approach value;
   setting the distance value to the maximum allowed approach value;
   generating the fast approach trajectory with a trapezoidal speed profile using the set speed and distance;
   controlling the motorbox axes to move using the generated trajectory, in which each axis stops when the respective load cell force thereof is obtained; the routine waits for all the axes to have completed the movement thereof; the distances not travelled of the excess trajectories are discarded;
j) checking that for each axis the distance travelled is less than the controlled trajectory distance;
k) checking that for each axis the force values detected by the load cells are those required;
l) if both of the above checks are passed, enabling the force control to maintain the same force on the piston motors of the instrument to compensate for an elongation or shortening of the tendons over time;
m) if the checks are not passed, command the axes to perform a disengagement routine;
n) when the user commands entering the teleoperation state, the current position of the motors is stored as kinematic zero.

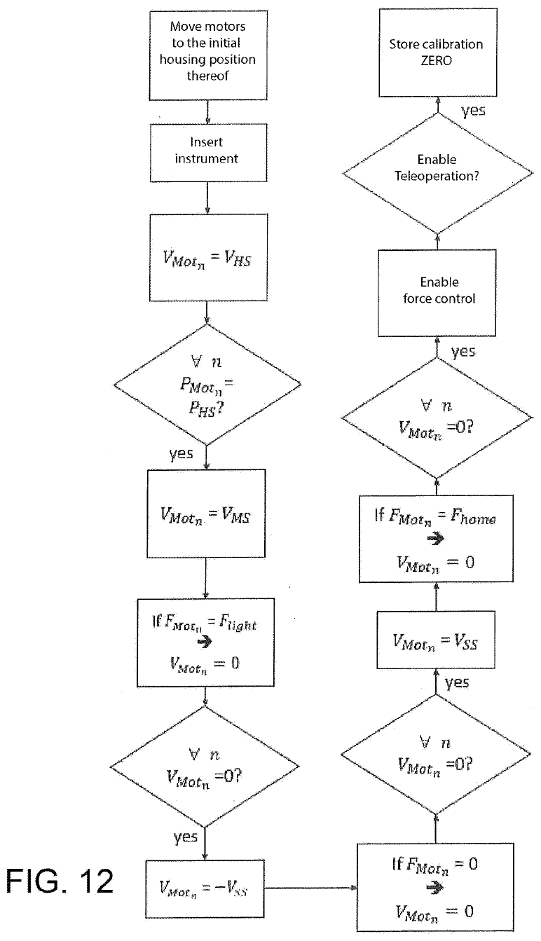

An implementation option of the method is shown in FIG. 12, in which the indicated parameters have the following meaning:
V_{Mot}_{ₙ} Speed of the n-th motor (motorized actuator);
P_{HS} Position of the motor (motorized actuator) to be reached with High Speed;
V_{MS} Medium Speed;
V_{HS} High Speed;
F_{Mot}_{ₙ} N-th motor force (motorized actuator);
F_{light} Light force;
V_{SS} Slow Speed;
Fₕₒₘₑ Engagement force.

According to an implementation option, the at least one actuator 11, 12, 13, 14, 15, 16 can be a linear actuator. The at least one transmission element 21, 22, 23, 24, 25, 26 can be a linear transmission element, such as a piston adapted to move along a substantially straight path x-x, as shown for example in FIG. 6.

To perform the calibration method, all the motorized actuators do not have to move simultaneously, although in accordance with a preferred embodiment the motorized actuators move (advance) simultaneously.

As shown diagrammatically for example in FIGS. 9A and 9B, a constraining body 37 or cap 37 can be fitted on the articulated end-effector 40 to lock one or more degrees of freedom P, Y, G, to facilitate the calibration procedure. A constraining body 37 can be provided to temporarily lock the articulated tip 40 in a predetermined configuration. The constraining body 37 can be retractable along the shaft 27 of the surgical instrument 20. The constraining body 37 can be a plug 37 or tip cap 37 which is not retractable along the shaft 27 of the surgical instrument 20 and for example can be removed distally with respect to the free end of the articulated end-effector 40.

The articulated end-effector 40 preferably comprises a plurality of links 41, 42, 43, 44, at least some of said links, for example the links 42, 43, 44 of FIG. 8, can each be connected to a pair of antagonistic tendons 31, 32; 33, 34; 35, 36.

As shown for example in FIG. 8, a pair of antagonistic tendons 31, 32 can be mechanically connected to a link 42 to move said link 42 with respect to a link 41 about a pitch axis P, in which the link 41 is shown integral with the shaft 27 of the surgical instrument 20; another pair of antagonistic tendons 33, 34 can be mechanically connected to a link 43 (shown here having a free end) to move said link 43 with respect to the link 42 about a yaw axis Y; yet another pair of antagonistic tendons 35, 36 can be mechanically connected to a link 44 (shown here having a free end) to move said link 44 with respect to the link 42 about a yaw axis Y; an appropriate joint activation of the links 43 and 44 about the yaw axis Y can determine a degree of freedom of opening/closure or grip G. Those skilled in the art will appreciate that the configuration of the tendons and the links as well as the degrees of freedom of the articulated end-effector 40 can vary with respect to that shown in FIG. 8 while remaining within the scope of the present disclosure.

Three pairs of antagonistic tendons (31, 32), (33, 34), (35, 36) can be present to actuate three degrees of freedom (e.g., the degrees of freedom of pitch P, yaw Y, and grip G). In such a case, the surgical instrument 20 can comprise six transmission elements 21, 22, 23, 24, 25, 26 (for example six pistons, as shown for example in FIG. 4), i.e., three pairs of antagonistic transmission elements (21, 22), (23, 24), (25, 26), intended for example to cooperate with three pairs of antagonistic motorized actuators (11, 12) (13, 14), (15, 16).

A sterile barrier 19 can be interposed between the at least one actuator and the at least one transmission element, such as a sterile cloth made as a plastic sheet or other surgically sterile cloth material, such as fabric or non-woven fabric.

The at least one tendon is preferably non-elastically deformable, although it can also be elastically deformable.

In accordance with a preferred embodiment, said at least one tendon and preferably all the tendons of the surgical instrument 20 are made of polymeric material.

Preferably, said at least one tendon, and preferably all tendons, of the surgical instrument 20, comprise a plurality of polymer fibers wound and/or intertwined to form a polymeric strand. In accordance with an embodiment, said at least one tendon comprises a plurality of high molecular weight polyethylene fibers (HMWPE, UHMWPE).

Said at least one tendon can comprise a plurality of aramid fibers, and/or polyesters, and/or liquid crystal polymers (LCPs), and/or PBOs (Zylon®), and/or nylon, and/or high molecular weight polyethylene, and/or any combination of the foregoing.

Said at least one tendon can be made of metal material, such as a metal strand.

Said at least one tendon can be partially made of metal material and partially of polymer material. For example, said at least one tendon can be formed by the intertwining of metal fibers and polymer fibers.

An electronic controller 9 of the robotic system 1, for example operatively connected to said at least one robotic manipulator 10, can monitor the movement of the actuators 11, 12, 13, 14, 15, 16 (e.g., motor pistons) and the calibration procedure can comprise bringing the actuators into contact with the respective transmission elements when the degrees of freedom of the articulated tip 40 of the surgical instrument 20 are in a predetermined configuration, for example the links of the articulated tip are aligned along the centerline of the instrument and/or the centerline r-r of the scope of each degree of freedom.

Such a predetermined condition can occur when the links of the articulated tip 40 are aligned with the stroke x-x of the transmission elements 21, 22, 23, 24, 25, 26.

Preferably, the electronic controller 9 is associated with a memory 8 for storing the zero position of the motorized actuators.

The zero position of the motorized actuators does not necessarily imply that the motorized actuators are all at the same level, in other words the transmission elements of the surgical instrument are not necessarily all at the same level within the respective stroke when the zero position is reached, as shown for example in FIGS. 10A-D. In fact, some polymeric tendons can have undergone a different elongation.

Referring again to FIGS. 1-15, a teleoperated robotic surgery system 1 is described, comprising a surgical instrument 20, a plurality of motorized actuators 11, 12, 13, 14, 15, 16 and further comprising control means 9.

The surgical instrument 20 comprises a plurality of transmission elements 21, 22, 23, 24, 25, 26 associated with a respective plurality of tendons 31, 32, 33, 34, 35, 36, and a articulated end-effector device 40, which is mechanically connectable through respective tendons to the transmission elements, so as to determine a univocal correlation between a set of movements of the transmission elements and a respective movement or pose of the articulated end-effector device 40.

The aforesaid articulated end-effector device 40 is adapted to be arranged and locked in a known predetermined position, considered as the reference position of the articulated end-effector device 40, in which such a reference position of the articulated end-effector device 40 is uniquely associated with a respective resulting position of each of the transmission elements 21, 22, 23, 24, 25, 26.

The motorized actuators 11, 12, 13, 14, 15, 16 are operatively connectable to respective transmission elements 21, 22, 23, 24, 25, 26 to impart movement to the transmission elements under control of the control means 9.

The control means 9, when the articulated end-effector device 40 is arranged and locked in said known predetermined position, considered as the reference position, are configured to perform the following actions:
actuating the motorized actuators 11, 12, 13, 14, 15, 16 so that each of the motorized actuators comes into contact with a respective transmission element 21, 22, 23, 24, 25, 26, controlling the motorized actuators 11, 12, 13, 14, 15, 16 so that they apply a force greater than zero and less than or equal to a threshold force on the respective transmission element of the surgical instrument;
storing the position of all the motorized actuators 11, 12, 13, 14, 15, 16 when each motorized actuator comes into contact with a respective transmission element, and considering the set of stored positions of the motorized actuators as the reference position of the motorized actuators univocally associated with the reference position of the end-effector device 40;
zeroing, i.e., defining a kinematic zero condition, associating the aforesaid stored reference position of the motorized actuators with a virtual zero point with respect to which the movements imparted by the control means 9 to the motorized actuators 11, 12, 13, 14, 15, 16 are to be referred.

According to different embodiments, the teleoperated robotic surgery system 1 is configured to perform a calibration method according to any of the method embodiments illustrated in this description.

As can be seen, the objects of the present invention as previously indicated are fully achieved by the method described above by virtue of the features disclosed above in detail, and as already disclosed above in the summary of the invention.

In order to meet contingent needs, those skilled in the art may make changes and adaptations to the embodiments of the method described above or can replace elements with others which are functionally equivalent, without departing from the scope of the following claims. All the features described above as belonging to a possible embodiment can be implemented irrespective of the other embodiments described.

### LIST OF REFERENCE SIGNS

| | |
|---|---|
| 1 | Robotic system for teleoperated surgery |
| 2 | Slave assembly of the robotic system |
| 3 | Master console |
| 8 | Memory |
| 9 | Controller, i.e., control unit |
| 10 | Robotic system manipulator |
| 11, 12, 13, 14, 15, 16 | Motorized actuators |
| 17, 17′, 18, 18′ | Force sensors, or load cells |
| 19 | Sterile barrier |
| 20 | Surgical instrument |
| 21, 22, 23, 24, 25, 26 | Transmission elements |
| 27 | Shaft |
| 28 | Pocket |
| 29 | Surgical instrument backend |
| 31, 32, 33, 34, 35, 36 | Tendons |
| 37 | Constraining body, or plug, or cap |
| 40 | Articulated tip, or articulated end-effector device |
| | of the surgical instrument |
| 41, 42, 43, 44 | Links of the articulated tip |
| 46 | Elastic element |
| x-x | Straight direction |
| r-r | Centerline |
| P, Y, G | Degree of freedom of the articulated tip, pitch, |
| | yaw, grip, respectively |

1. A method for calibrating a surgical instrument of a teleoperated robotic surgery system,
wherein the surgical instrument comprises a plurality of transmission elements associated with a respective plurality of tendons, and an articulated end-effector device, which is mechanically connectable through respective tendons to the transmission elements, to determine a univocal correlation between a set of movements of the transmission elements and a respective movement or pose of the articulated end-effector device,
wherein the teleoperated robotic surgery system further comprises, in addition to said surgical instrument, a plurality of motorized actuators and a controller, the motorized actuators being operatively connectable to respective transmission elements to impart a movement to the transmission elements controlled by the controller;
wherein the method comprises the steps of:
arranging and locking the articulated end-effector device in a known predetermined position, considered as the reference position of the articulated end-effector device, wherein said reference position of the articulated end-effector device is univocally associated with a respective resulting position of each of the transmission elements;
actuating the motorized actuators so that each of the motorized actuators contacts with a respective transmission element;
storing the position of all the motorized actuators when each motorized actuator comes into contact with a respective transmission element, and considering the set of stored positions of the motorized actuators as the reference position of the motorized actuators univocally associated with the reference position of the end device,
defining a kinematic zero condition, by associating said stored reference position of the motorized actuators with a virtual zero point with respect to which movements imparted by the controller to the motorized actuators are to be referred;
wherein said actuating step comprises controlling the motorized actuators so that the motorized actuators apply a force greater than zero and less than or equal to a threshold force on the respective transmission element of the surgical instrument.

2. A method according to claim 1, wherein said threshold force is predetermined in a preliminary step of determining a threshold force, to impart a preload to the tendons operatively connected to both the transmission elements and the articulated end-effector device, under conditions in which the end-effector device is held still and locked,
and wherein said actuating step comprises controlling the motorized actuators so that the motorized actuators apply a force equal to said threshold force on the respective transmission element of the surgical instrument, within a tolerance.

3. A method according to claim 1, wherein the teleoperated robotic surgery system comprises force sensors, each force sensor being operatively connected to a respective transmission element, and/or
wherein the motorized actuators are configured to apply the force to respective transmission elements and detect the force actually applied to each transmission element, and
wherein said step of applying a force greater than zero and less than a threshold force on each transmission element comprises applying a force to the transmission element by a feedback control loop, wherein a feedback signal is representative of the force applied to the transmission element as actually detected by the respective force sensor operatively connected to the transmission element or to the respective motorized actuator.

4. A method according to claim 1, wherein said articulated end-effector device comprises joints, and
wherein said predetermined known position of the articulated end-effector device is:
a position corresponding to the condition in which each joint of the articulated end-effector device is in a centered position of the joint workspace thereof, or
a position corresponding to the condition in which the articulated end-effector device is aligned with the axis of a shaft of the surgical instrument.

5. (canceled)

6. A method according to claim 1, wherein the reference position of the articulated end-effector device is held constrained by a tip cap.

7. A method according to claim 1, wherein said threshold force, at which the motors of the motorized actuators stop in contact with the respective transmission elements, is in a range of 0.01 N to 5.0 N.

8. A method according to claim 1, wherein a control of an offset between the reference position of the motorized actuators, and of each of the motorized actuators, independently of the others, and a predetermined nominal zero position is carried out, and if the offset is greater than a maximum allowable absolute offset, the calibration procedure is considered invalid, or
wherein a control of the relative offset between the positions reached by each motorized actuator when in contact with the corresponding transmission element is carried out, and if the relative offset is greater than a maximum allowable relative offset, the calibration procedure is considered invalid.

9-10. (canceled)

11. A method according to claim 1, wherein one or more pairs of antagonistic transmission elements are provided, being operatively connectable to respective one or more pairs of antagonistic tendons, each pair of antagonistic tendons being adapted to move a single-piece link of the articulated end-effector device in opposite movement directions.

12. A method according to claim 1, wherein elastic elements are provided, which act on respective transmission elements to keep a constant minimum preload level adapted to space apart the transmission elements from the respective motorized actuators.

13. A method according to claim 1, wherein said actuating step comprises controlling the motorized actuators so that, in a first contact step between motorized actuators and respective transmission elements, a first speed is imparted to the motorized actuators and a first force is applied on the respective transmission elements.

14. A method according to claim 13, wherein said actuating step comprises controlling the motorized actuators so that said first speed is in a range of 0.1 to 30 mm/s, and/or
to stop the movement of said motorized actuators when said first force is detected to be in a range of 0.01 to 2 N.

15. A method according to claim 13, wherein said actuating step comprises, in addition to said first contact step:
a retraction step, in which the motorized actuators retract by a shift,
a second advancement and second contact step, in which the motorized actuators advance at a second speed and stop when a contact force equal to a second force is detected.

16. A method according to claim 15, wherein the second force is equal to said threshold force, or
wherein said second speed is lower than said first speed;
and/or wherein said second force is greater than said first force.

17. (canceled)

18. A method according to claim 15, wherein, in said retraction step, the movement of the motorized actuators is controlled so that the force applied by the motorized actuators reaches a third force value,
wherein said third force value is in a range of 0.1 to 5 N.

19. A method according to claim 13, wherein said actuating step comprises controlling the motorized actuators so that the motorized actuators advance at a speed equal to a third speed, greater than said first speed and second speed, when the position of the motorized actuators is in a predetermined range in which the controller knows that a free stroke regime is occurring, prior to the first contact with the transmission elements, along a space corresponding to a stroke.

20. A method according to claim 1, wherein a flexible and elastic sterile drape is interposed between said motorized actuators and said surgical instrument, and
wherein the force generated by the resistance of said sterile drape is a known off-set or bias force, and wherein the controller is configured to take into account, or to remove or not consider, said known off-set or bias force from the force checks carried out, and/or from comparison with the threshold force.

21. A method according to claim 1, wherein the controller moves the articulated end-effector device, when the articulated end-effector device is in the condition to move without being locked by external constraints, applying a maximum operating force,
wherein said maximum operating force is less than or equal to said threshold force, or
wherein the motorized actuators comprise pistons and/or rotary discs.

22. (canceled)

23. A method according to claim 1, wherein the antagonistic tendons are operatively connected to both respective transmission elements and respective links of the articulated end-effector device to actuate, with opposite movements, at least one degree of freedom among said at least one degree of freedom of the articulated end-effector device,
wherein, after the step of contact or engagement between motorized actuators and transmission elements, the defining step is carried out simultaneously on the antagonistic tendons of a pair of agonistic-antagonistic tendons for each degree of freedom of the end device, and,
said defining step is applied in sequence to the pairs of antagonistic tendons, or is carried out for one pair at a time; or
wherein the antagonistic tendons are operatively connected to both respective transmission elements and respective links of the articulated end-effector device to actuate, with opposite movements, at least one degree of freedom of said at least one degree of freedom of the articulated end-effector device,
wherein, after the step of contact or engagement between motorized actuators and transmission elements, the defining step comprises, for each of the controlled degrees of freedom of the end-effector device;
bringing each of the degrees of freedom of the end-effector device to an end-of-stroke abutment,
applying a high force to the respective transmission element, thus stressing the respective tendon;
storing, for each of the degrees of freedom, the corresponding position of the transmission element which is thus obtained;
defining and/or recalculating the kinematic zero position based on the stored positions of the transmission element for each of the degrees of freedom;
wherein, said bringing, applying, storing, and defining and/or recalculating steps are carried out for all the transmission elements, for the transmission elements and the mutually antagonistic tendons, so that for each degree of freedom, the two positions of the two transmission elements associated with the antagonistic tendons of said degree of freedom are stored.

24. (canceled)

25. A method according to claim 1, wherein an angular distance between the kinematic zero position of a degree of freedom and an end-of-stroke thereof is known, and wherein the defining step comprises:
bringing one degree of freedom of the end-effector device to the stroke end abutment,
bringing the force acting on a tendon of a pair of antagonistic tendons to a high force value;
storing the position of the transmission element corresponding to said tendon;
keeping the high force applied to said tendon, while the step of applying an antagonistic force to the other tendon of the pair of antagonistic tendons is carried out, wherein said high force is greater than said antagonistic force;
storing the position of the transmission element corresponding to said antagonistic tendon;
calculating the kinematic zero position of the antagonistic transmission elements of said pair of antagonistic transmission elements based on the stored values of the respective positions;
moving said transmission elements to the calculated kinematic zero position.

26. A method according to claim 1, wherein said tendons are polymer tendons, made of intertwined polymer fibers.

27. A teleoperated robotic surgery system comprising a surgical instrument, a plurality of motorized actuators and a controller,
wherein the surgical instrument comprises a plurality of transmission elements associated with a respective plurality of tendons, and a articulated end-effector device, which is mechanically connectable through respective tendons to the transmission elements, to determine a unique correlation between a set of movements of the transmission elements and a respective movement or pose of the articulated end-effector device,
wherein said articulated end-effector device is adapted to be arranged and locked in a known predetermined position, considered as a reference position of the articulated end-effector device, wherein said reference position of the articulated end-effector device is univocally associated with a respective resulting position of each of the transmission elements;
wherein the motorized actuators are operatively connectable to respective transmission elements to impart movement to the transmission elements under the control of the controller;
wherein the controller, when the articulated end-effector device is arranged and locked in said known predetermined position, considered as the reference position, is configured to:
actuate the motorized actuators so that each of the motorized actuators comes into contact with a respective transmission element, controlling the motorized actuators so that the motorized actuators apply a force greater than zero and less than or equal to a threshold force on the respective transmission element of the surgical instrument;
store the position of all the motorized actuators when each motorized actuator comes into contact with a respective transmission element, and consider the set of stored positions of the motorized actuators as the reference position of the motorized actuators univocally associated with the reference position of the end-effector device;
define a kinematic zero condition by associating said stored reference position of the motorized actuators with a virtual zero point with respect to which the movements imparted by the control means to the motorized actuators are to be referred.

28. (canceled)

29. A method according to claim 1, wherein said threshold force, at which the motors of the motorized actuators stop in contact with the respective transmission elements is in a range of 0.05 N to 2.0 N.

30. A method according to claim 13, wherein said actuating step comprises controlling the motorized actuators so that said first speed is between 1 and 10 mm/s, and/or
to stop the movement of said motorized actuators when said first force is detected to be in a range of 0.05 N to 0.5 N.

31. A method according to claim 15, wherein the second force is equal to said threshold force, or
wherein said second speed is lower than said first speed, and in a range of 0.1 to 5 mm/s; and/or
wherein said second force is greater than said first force, and in a range of 0.1 to 5N.

32. A method according to claim 15, wherein the second force is equal to said threshold force, or
wherein said second speed is lower than said first speed, and in a range 0.5 to 3 mm/s; and/or
wherein said second force is greater than said first force, and in a range of 0.5 to 2N.
